(19) United States
(12) Patent Application Publication
(10) Pub. No.: US 2017/0086941 A1
(43) Pub. Date: Mar. 30, 2017

(54) Marker for Optical Tracking System

(71) Applicant: Atracsys, Puidoux (CH)

(72) Inventors: Gaëtan Marti, Epalinges (CH); Maurice Hälg, Lausanne (CH)

(21) Appl. No.: 15/273,796

(22) Filed: Sep. 23, 2016

Related U.S. Application Data
(60) Provisional application No. 62/232,597, filed on Sep. 25, 2015.

(51) Int. Cl. A61B 90/00; A61B 34/20

(52) U.S. Cl. CPC A61B 90/39; A61B 34/20; A61B 90/36; A61B 2034/2055; A61B 2090/3983; A61B 2090/3945

(57) ABSTRACT

A marker including a support with a front surface and a back surface, a light generating element fixed on the back surface of the support, and a device for fixation of the light generating element on the back surface, wherein the support includes at least three openings placed at predetermined positions on the support, wherein the light generating element is fixed on the back surface of the support by the device for fixation to cover the openings such that the light generating element is detectable through the openings by the tracking system, and wherein the light generating element can be placed or removed or replaced during an operation with the device for fixation.

### CROSS-REFERENCE TO RELATED APPLICATION

The present application claims priority to earlier U.S. provisional application with the Ser. No. 62/232,597 filed on Sep. 25, 2015, the content of this earlier application being incorporated in its entirety by reference in the present application.

### TECHNICAL FIELD

The present invention is directed to the field of optical tracking systems and of markers used in such systems. Typically, such optical tracking systems are used in the field of computer assisted surgery or dentistry, robotic assisted surgery or dentistry, rehabilitation and other similar medical applications. On a more general level, they are used in the field of motion capture, that can be linked to medical applications or other applications where the tracking of movements is required.

### BACKGROUND ART

Traditional optical pose tracking systems comprise two cameras. They use triangulation to determine the three-dimensional (3D) position of light generating elements in space. These light generating elements may either be active: they transmit light (e.g. LEDs) or passive: they reflect light (e.g. reflective disks or spheres), or a combination of active and passive. In the passive case, the light used to be generated on rings of LEDs located around the cameras of the optical tracking system. Emitted light is further reflected on tracked objects and sensed by the cameras.

Such a tracking system is able to compute (that is determine) the 3D position of a single light generating element. If at least 3 light generating elements with known relative positions are measured, it is further possible to compute (that is determine) a 3D orientation. Such an object is called a marker and markers are fixed on target objects/subjects to be tracked.

In a medical application context, a user (e.g. a surgeon) touches a region of interest on the patient's body using a distal tip of an object (e.g. a probe or a surgical instrument). A tracking system views the marker(s) affixed to the object and is able to retrieve its/their pose(s). Then, on the basis of the known relationship between the location of the marker(s) and the location of the object tip, the marker-tracking system determines the coordinates of the object's tip as well as its orientation. Such pieces of information can be used for example by an application to register the patient with a preoperative planning and/or to steer the interventional radiologist when inserting a biopsy needle.

Moreover, medical and especially surgical applications require the markers to be sterile. Common passive markers are usually disposable, autoclavable or a mix of the two.

In U.S. Pat. Pub. No. 2007/0183041, markers include light generating elements comprising two spherical caps of different radii that are disposed substantially concentric in relation to one another. Such technique enables to manufacture disposable markers as the Radix™ from Northern Digital™.

Most common reflective markers are using glass bead technology which embed numerous tiny glass beads in a reflective substrate (e.g. the 3M™ Scotchlite™ Reflective Material). The reflective foil is either taped on flat disks or stretched on spheres. U.S. Pat. No. 6,675,040 presents numerous different naive constructions of such markers. A more elaborated construction is described in U.S. Pat. No. 8,386,022, presenting a multi-face disposable marker comprising reflective disks.

The highly-textured surface of a reflective foil is susceptible to contamination from dirt, blood, fingers, oils, etc. Any contamination that is present on the surface of the marker may affect the retro-reflective performance of the passive marker, thereby contributing or causing inaccuracies in the determination of a position of the object to which the marker is affixed. Therefore, the part containing the reflective material should preferably be easily exchanged and ideally disposable for the sake of safety and simplicity. German Patent Publication DE102008022254A1 presents an exchangeable light generating element which further embed the reflective material within a dome to reduce the contamination problem.

International Patent Publication WO2014/127814A1 presents a marker with a recess mechanism to exchange the light generating element at each operation. The base of the marker is autoclave for further use. This product unfortunately comprises several compartments and recesses which are difficult to clean during the sterilisation process. There is moreover a high probability to damage/scratch the reflecting material when inserting the light generating element in the recess. This will have a high impact on the tracking precision and at the end on the security for the patient.

In light of all the drawbacks and deficiencies of the background art, more advanced and innovative devices and systems for markers are desired, for various tracking applications.

### SUMMARY

The invention generally relates to a low-cost marker for optical tracking which includes a marker, one or several light generating elements and a mechanism that enable to easily fix/exchange the light generating element on the markers.

There has thus been outlined, rather broadly, some of the features of the invention in order that the detailed description thereof may be better understood, and in order that the present contribution to the art may be better appreciated. There are many additional features of the invention that will be described hereinafter.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

According to one aspect of the present invention, a low-cost marker for optical tracking is provided, preferably comprising at least two distinct parts, the first one providing a support with openings that are precisely placed and the second one comprising one or several light generating elements (reflectors, LEDs, salient points, etc.) that are fixed under the support, facing the optical tracking system.

Another object of the present invention is to provide a low-cost marker for optical tracking that is intended to be used during computer assisted surgery (respectively dentistry, or interventional radiology) operations together with a passive near-infrared optical pose tracking system.

Another object of the present invention is to provide a low-cost marker for optical tracking that could be used for any pose tracking application including motion capture, rehabilitation, gait analysis, quality control or reverse engineering or any other tracking application.

Another object of the present invention is to provide a low-cost marker for optical tracking that is cheap enough to be manufactured as a completely or partially disposable.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application, for example by using equivalent means.

In one embodiment, a marker for an optical tracking system is provided, wherein said marker comprises at least a support with a front surface and a back surface, wherein said support comprises at least three openings placed at predetermined positions on said support wherein one or several light generating element(s) is/are fixed on the back surface of the support, covering the openings such that the light generating element(s) are detectable through the openings by said tracking system, and means for maintaining said light generating element on said back surface and wherein the light generating elements can be placed or removed or replaced during the operation. The operation may by any tracking application using the present marker. It may by a surgical operation or another application.

In one embodiment, the openings of the maker are circular with a chamfer being located around the openings where its sharp part is close to the light generating element in order to reduce the tracking error when the marker is angulated.

In one embodiment, a light generating element covers at least one of said openings.

In one embodiment, the support comprises one light generating element per opening.

In one embodiment, the fixation of the light generating element(s) to the support of the marker is realized by a spring mechanism or another equivalent mechanism.

In one embodiment, the spring mechanism comprises at least a leaf spring.

In one embodiment, the fixation of the light generating element(s) to the support of the marker is realized by magnetic means, for example a permanent magnet.

In one embodiment, the fixation mechanism is located on the marker or support.

In one embodiment, the fixation mechanism is located on light generating element(s).

In one embodiment, the fixation mechanism is partly located on light generating element(s) and partly on the support.

In one embodiment, the light generating elements comprise a reflective foil.

In one embodiment, the foil is applied on the back surface of the support and covers at least the openings.

In one embodiment, the foil is maintained on said back surface by a lower marker part which is held by attachment parts.

In one embodiment, the light generating element comprises a diffuser and a light source.

In one embodiment, the light source comprises a Light Emitting Diode (LED) mounted on a Printed Circuit Board (PCB).

In one embodiment, a tracking system comprising at least one marker as defined herein is provided.

In one embodiment, the tracking system comprises at least two markers as defined herein and said system further comprising camera means tracking the markers.

In one embodiment of the tracking system, the camera means comprise two cameras and illuminating means to illuminate said reflective foil.

In one embodiment of the tracking system the illuminating means comprise a ring of LED surrounding each camera.

In one embodiment, an image guided surgery system comprising at least a marker as defined herein or a tracking system as defined herein is provided. The system may comprise a robotic element, such as a robotic arm, used in the operation.

### BRIEF DESCRIPTION OF THE SEVERAL VIEWS OF THE DRAWINGS

Various other objects, features and advantages of the present invention will become fully appreciated as the same becomes better understood when considered in conjunction with the accompanying drawings, in which like reference/characters designate the same or similar parts throughout the several views, and wherein:

FIG. 1 illustrates a perspective view of an embodiment of a low-cost marker according to one aspect of the present invention. It comprises at least two distinct parts, the marker 10 and a light generating element 20. In this embodiment, the marker 10 could be a planar support that has a mechanism 11 to attach it on an object/subject. The marker contains multiple openings 12 that are precisely placed. A chamfer located around the openings enable to improve the tracking accuracy when the marker is rotated with respect to the tracking system. Light generating elements 20 have a diameter slightly larger than the openings 12. They fit in the openings from the backside, facing the tracking system, and are blocked with leaf springs 13 as attachment means;

FIG. 2 illustrates a partial sectional view of the marker 10 of FIG. 1 including the light generating element 20 and the leaf spring 13 means;

FIG. 3 illustrates another embodiment of the marker which is comprises three distinct parts: The upper marker 10 with the support and the openings 12 precisely placed, the attachment mechanism 11 to attach it on an object/subject, and additional attachment means 14, 31 to fix the upper 10 and lower marker 30 parts. A reflective foil 21 is placed in sandwich between the two marker parts (upper and lower). Finally, reference 31 identifies the clip matching part enabling the upper and lower marker parts 10, 30 to attach to each other and to lock the reflective foil 21 between them. Note that the reflective foil 21 can alternatively be glued on the lower marker 30;

FIG. 4 illustrates another embodiment of the present invention. Partial sectional view of the marker 10 of FIG. 1 including the reflective disk 20 with another fixation mechanism. In this embodiment, the disk comprises ferromagnetic parts and magnets 15 allowing to fix the disk directly on the marker (in replacement of the leaf springs 13 illustrated in FIG. 1). Alternatively, the magnet could be part of the disk and the ferromagnetic part located on the marker;

FIG. 5 illustrates an example of an application of the system according to another aspect of the present invention in combination with a passive optical tracking system 40 comprising two cameras 41, the cameras being surrounded by a ring of infrared LEDs 42 used to illuminate the light generating elements 20 on the markers 10. The left marker 10—for example one presented in FIG. 1—is fixed on the object/subject to be tracked 60. The right marker 10 is fixed on (resp. part of) a probe 51 held by a user 50 of the tracking system, for example a surgeon. The user 50 of this FIG. may also illustrate a robotic element, such as a robotic arm resp. hand;

FIG. 6 illustrates another embodiment of the present invention with a sectional view of an active marker 10. Contrary to FIG. 1, the reflective disk 20 is replaced by a diffusing material 22 fixed on the support. In this embodiment, light is not reflected but is generated by the underlying LED 23. The LED is for example fixed on a PCB including control circuits and a battery. The PCB is fixed to the upper part of the marker using leaf springs 13 as an example;

FIG. 7 illustrates another embodiment of the present invention with a perspective view of a marker 10 comprising a support 17 having a front surface 18 and a back surface 19. The openings 12 are precisely placed on the support 17. They are surrounded by a chamfer 16 to improve the tracking accuracy when the marker is rotated. Light generating elements 20 have a diameter slightly larger than the openings 12. They fit in the openings from the back surface. When in place, there are facing the tracking system (for example as illustrated in FIG. 5). Fixation means 24 are in this embodiment located on the light generating element part enabling to clip them on the marker; and

FIG. 8 illustrates an alternative embodiment of FIG. 7. The clipping mechanism is replaced by a fixation mechanism 25 based on a leaf spring protruding from the side of the disk 20. When inserted in the back of the marker the lateral leaf spring will block the light generating element 20. A groove can alternatively be machined on the backside of the marker 19 and around the opening 12 in order to secure the light generating elements 20 in a stable and known position.

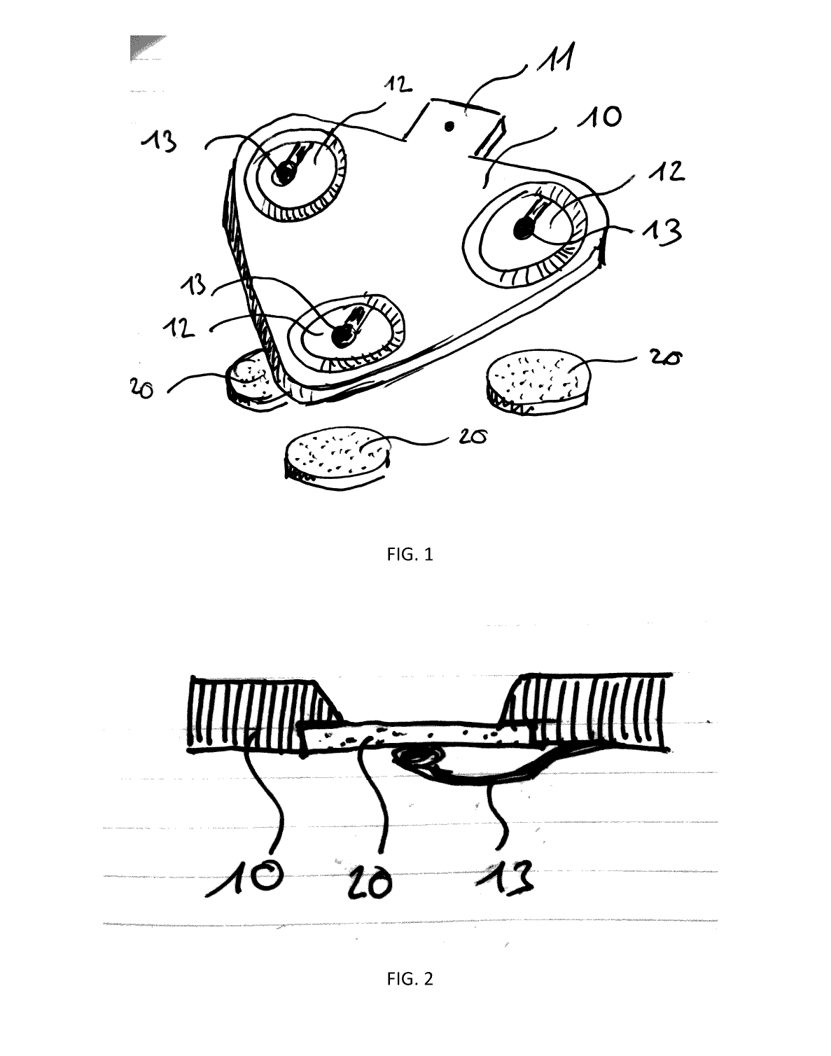

### DETAILED DESCRIPTION OF THE SEVERAL EMBODIMENTS

Turning now descriptively to the drawings, in which similar reference/characters denote similar elements throughout the several views, the figures exemplary illustrate a marker, one or several light generating elements (e.g. reflective parts, LEDs) and a mechanism system that allow to fix the light generating elements part(s) on the marker.

### Marker

The marker comprises a support 17 and an attachment mechanism 11. The support 17 contains several openings 12, designed such as the light generating element 20 is visible through an opening and is ideally flat and circular.

The openings 12 should be very precisely tooled such that the visible part of the light generating element 20 seen through them is reproducible. This is especially interesting when manufacturing a batch of markers as it will avoid calibrating them individually.

The openings 12 should preferably have a chamfer 16 that will enable to see the light generating element 20 when angulating the marker 10 with respect to the tracking system without losing tracking accuracy.

The marker 10—also called rigid body in the literature—comprises several light generating elements 20 rigidly fixed together. The position of the light generating elements 20 with respect to the attachment mechanism 11 is called the geometry and should be well known. During the tracking process, these light generating elements 20 are detected in 3D by the tracking system. If at least three fiducials 20 are detected, an algorithm is further used to calculate the pose (position+translation) of the marker 10 in the tracking system referential. For example, the algorithms could be the ones described in Arun K. Somani et al., “Least-squares fitting of two 3-D point sets,” IEEE Transactions on pattern analysis and machine intelligence 5, 1987, pp. 698-700, this reference herewith incorporated by reference in its entirety. The precision of this step is partly defined by the precision of the geometry, which is influenced by the manufacturing process of the support. Preferably, the proposed marker 10 has circular openings 12—very precisely manufactured—under which the light generating element(s) 20, 21 is/are located so that the support acts as a mask. A chamfer 16 on one side of the opening facing the tracking means (e.g. cameras) avoids partial occlusion by enabling the tracking system to see the entire light generating element even if the openings 12 are not parallel to the cameras of the tracking system.

The marker 10 may be manufactured out of metal, carbon and/or hard plastic part or a mix therefrom. It may have the fixation mechanism 13, 15 directly integrated in the marker. It may be designed to be autoclaved or disposable for surgical applications. A marker may be directly integrated in the design of a probe/tool 51. Ideally, the support of the markers is precisely tooled and/or molded to avoid an individual calibration process.

### Light Generating Elements

The light generating elements 20 may be active (such as a LED) or passive (such as a reflective material). The visible surface (seen by the tracking system through the openings 12) is fixed on the back surface of the support 10. These openings 12 are slightly smaller than the reflective material and thus acts as masks. Alternatively, the fixation mechanism 30, 24, 25 could be part of the light generating element.

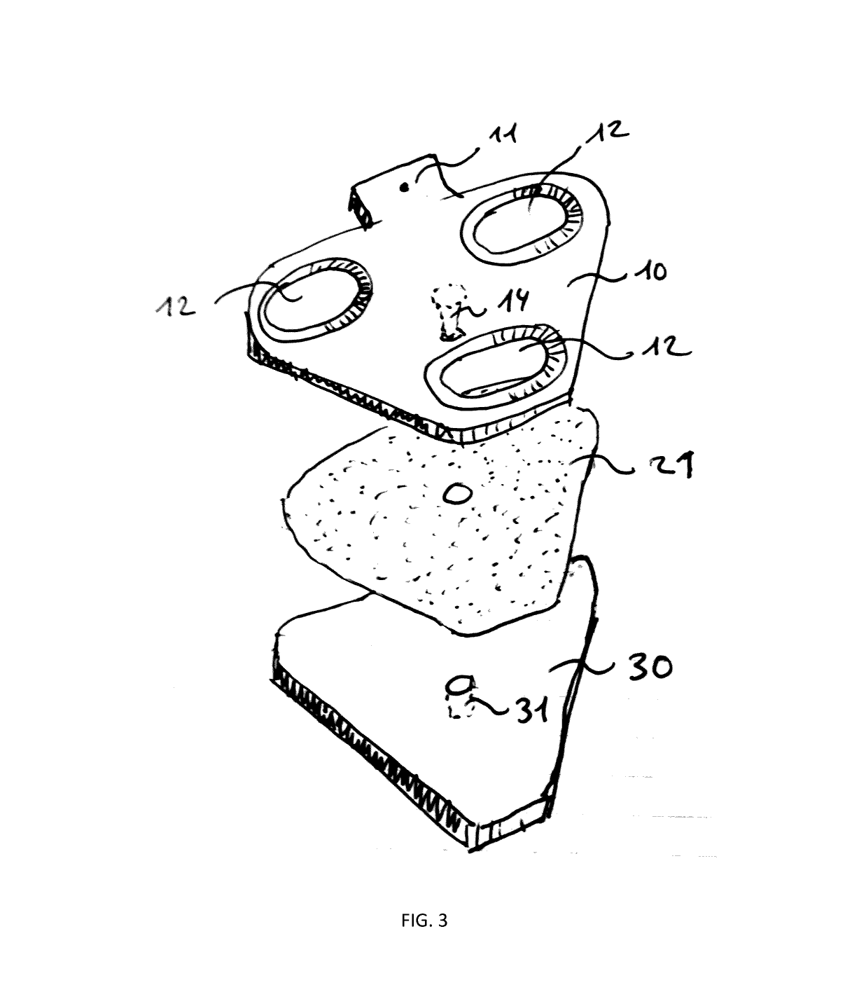

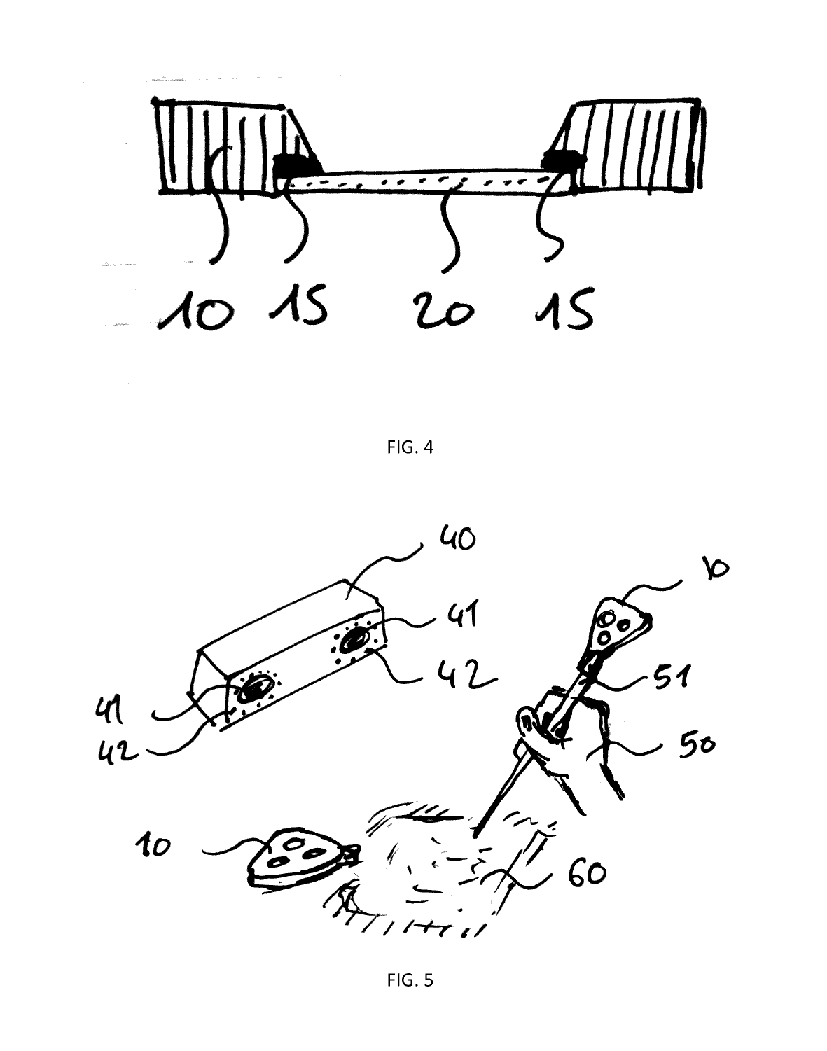

In case of passive elements, the reflective material is optimized to work in the wavelength specified by the tracking system (e.g. near infrared). The reflecting surface should be flat, as uniform as possible, and should give a good response with different angulations of the marker with respect to the tracking system. Ideally, the reflecting surface is larger than the hole 12 of the marker 10 so that it may be easily adjusted. The reflective part may be a disk 20 (e.g. shape and size of a flat coin) with a reflective foil 21 taped on it. The reflective part 20 may have magnetic parts in it or being made out of ferromagnetic material so that it may be magnetically clipped to the marker 15. Alternatively, the reflective part may just be a foil 21, as described in FIG. 3. As the reflective material is very delicate and can be damaged by liquids, scratches and/or occlusions, it is necessary to be able to easily exchange the reflective parts 20 or foil 21 before or during an operation. Note that in surgical applications, existing reflective material cannot be sterilized with autoclave. It is thus necessary to have this part disposable.

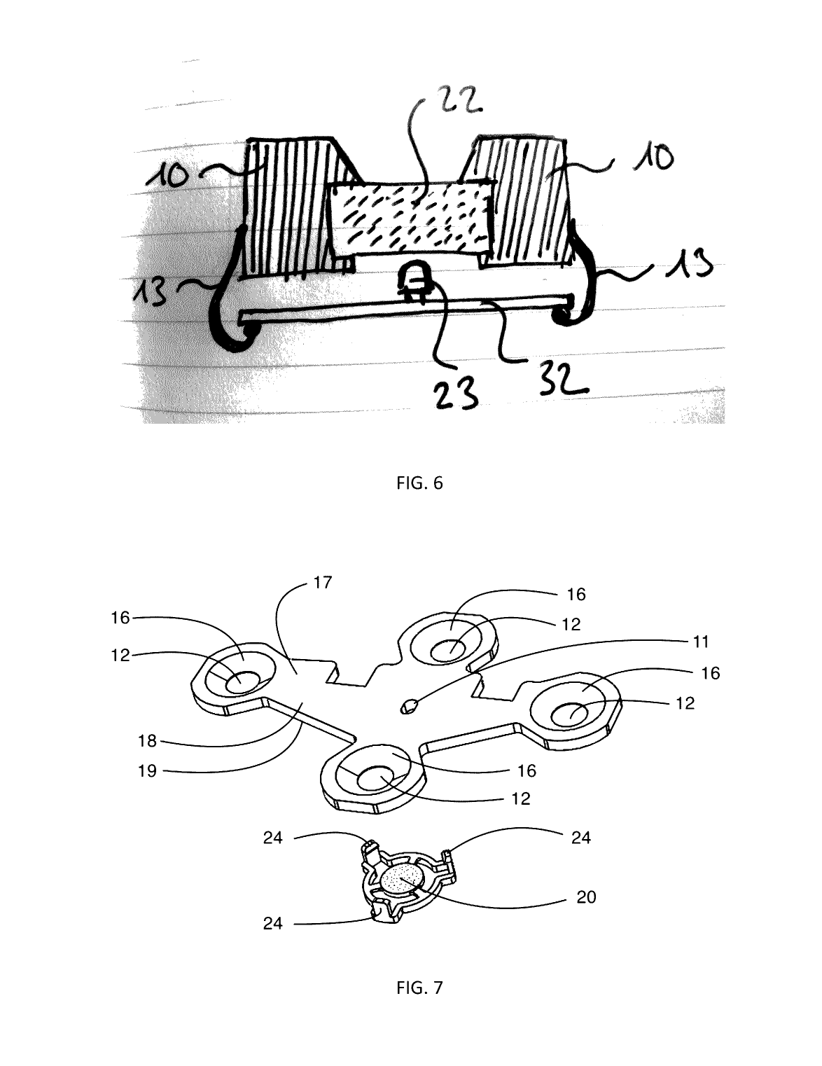

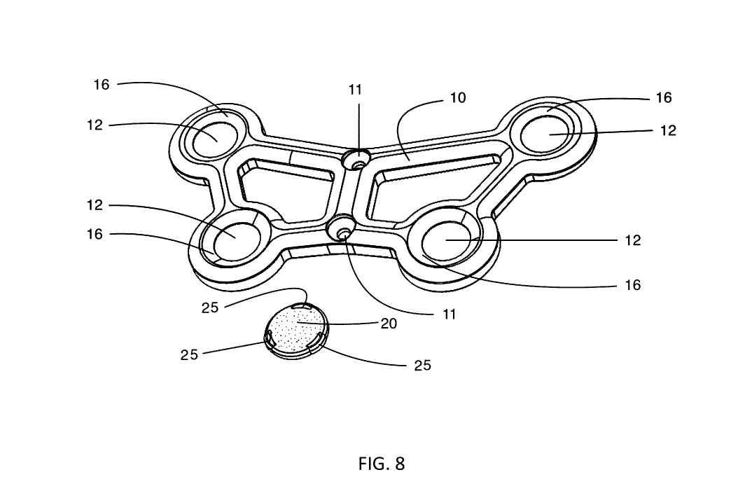

In case of an active element—as shown for example in FIG. 6—the reflective part is replaced by a diffusing material 22 fixed on the back surface of the support. Light is not reflected but is generated by an underlying LED 23. This mechanism has the advantage of not requiring a very precise placement of the LED on the PCB as the diffuser will create a uniform illumination and the opening a precise placement.

### External Fixation Mechanism

The fixation mechanism enables to fix or attach the light generating element part on the marker. This fixation could be part of the marker 10 (e.g. the leaf springs 13 or magnetic fixation 14). The fixation could alternatively be placed on the light generating element (e.g. the leaf spring 25 or clipping system 24). Finally, the fixation could be partly on the marker and partly on the light generating element (e.g. the clipping system 14). A combination of the fixations is also possible.

The fixation is designed to attach the light generating element 20 to the back of the support's opening 12. It should also enable the light generating element 20 to be easily exchanged at any time (e.g. during a surgical intervention if it is occluded by body fluids for example).

The fixation mechanism may be a clip 24, 31, a magnetic clip 15, or a leaf spring 13, 25. Any other equivalent type of fixation mechanism is of course possible as are combinations. As the marker 10 could be used multiple times unlike the reflective parts 20, the fixation mechanism should preferably prevent or at least minimize any damage to the marker 10.

### Passive Optical Tracking System

The Passive optical tracking systems comprise one or more cameras 41. They emit infrared light for example from a ring 42 surrounding each infrared camera lens and use spheres or discs as passive targets. Light generating elements are coated with a retro-reflective material containing small spheres that mirror the light back to the lenses, causing the light generating elements to appear as bright spots in the infrared images. Passive tracking systems can also detect light generating elements composed of infrared LEDs. Passive optical tracking systems can operate in visible and near IR spectra.

Passive optical tracking systems are widely used in surgery and motion capture applications. They basically comprise a set of camera with a known baseline—respective pose of the cameras. The cameras operate in the infrared so that the flashes are not disturbing the users. The flashes are emitted by infrared lights that are preferably arranged on a ring around the camera lenses.

Most commonly used camera are stereo-camera—with two cameras—fixed on a bar. The relative pose of the camera is factory calibrated. Once a light generating element is simultaneously detected by both cameras, it is possible to compute its 3D position by means of triangulation.

Example of commonly used passive optical tracking systems are Atracsys™ infiniTrack™ and fusionTrack™, NDI Polaris™ or Axios™ CamBar™. Any general purpose camera(s) can alternatively be used as passive optical tracking system. They generally are less precise than the ones comprising several cameras.

### User

In an embodiment of the application, a user 50 is handling a probe 51. The marker 10 according to the present invention is fixed on (respectively part of) a probe 51 or tool. The user 50 (e.g. a surgeon) is handling the probe such that the reflective parts are visible by the cameras during tracking. A user could alternatively be a robot or another equivalent device.

### Measuring Object/Subject

In an embodiment of the invention, a marker 10 is fixed on an object (respectively a subject) to be tracked 60. In a surgical application, the subject is typically the patient to be operated. The marker 10 may be fixed on a bone structure (femur, tibia, pelvis, skull, etc.), a head frame used in neurology, or screwed, glued, fixed on the patient via an elastic band, glasses' frame, etc.

### Operation of a Preferred Embodiment

FIG. 5 illustrates a typical computer assisted surgical setup using one or more markers 10. This setup comprises two markers 10. The first one is integrated in a probe 51 held by a user 50. The other one is fixed on the patient 60, for example but not limited via a screw fixed on a bone structure of the patient.

Prior to the surgical intervention, light generating elements 20, 21 have been fixed on the two distinct markers 10 by means of a fixation mechanism 13, 14, 15, 24, 25 as described above. Both markers 10 have a distinct geometry (relative pose of the openings) so that each can be uniquely detected by the system. The placement of the light generating elements 20 does not need to be precise but should perfectly fit the openings 12 of the marker 10 for a proper detection.

During the registration step, the surgeon will point specific anatomical structures by means of the probe 51 to create a matching between the pre-operative planning and the real patient. The reference marker on the patient is necessary to track and measure possible displacements of the patient and/or the passive tracking system during the assisted procedure.

During this step, at least three light generating elements 20 of each marker 10 should be visible by the tracking system 40. The tracking system 40 will perform image processing of the camera images to detect the center of gravity of the light generating elements 20, matching, triangulation and finally pose estimation given the unique geometry of the markers 10. The system will finally know the pose of the probe with respect of the marker 10 attached on the patient 60.

At the end of the surgical operation, the light generating elements 20 are thrown away. The marker 10 on the patient and the probe 51 are either designed to be autoclaved for further use or disposed.

Compared to existing technology (reflecting spheres), the proposed system according to the present invention has the advantage of being more precise because the precision does not rely on screwed or plugged spheres but only on the manufacturing of openings 12 which is by nature more precise. This will also reduce the operating costs as the reflective parts 20 are cheaper to manufacture compared to reflective disks. Finally, if the fixation mechanism is simple, ergonomics is improved because replacement of a reflective part during surgical procedure does not require a recalibration or reregistration step (which is required by existing systems).

What has been described and illustrated herein is a preferred embodiment of the invention along with some of its variations. The terms, descriptions and FIG.s used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

### INDEX OF ELEMENTS

10: Marker
11: Attachment Mechanism
12: Openings Within The Support
13: Fixation Mechanisms
14: Alternate Clip Fixation Mechanism
15: Alternate Magnetic Fixation Mechanism
16: Chamfer Around The Openings
17: Support
18: Front Surface (Of The Support)
19: Back Surface (Of The Support)
20: Light Generating Element
21: Reflective Foil
22: Diffusing Material
23: LED
24: Alternative Clip Fixation Mechanism
25: Alternative Leaf Spring Mechanism
30: Second Marker Part
31: Clip Hole
32: PCB
40: Passive Optical Tracking System
41: Camera
42: Led Ring
50: User
51: Probe/Tool
60: Measuring Object/subject

1. A marker for an optical tracking system comprising:
a support with a front surface and a back surface;
a light generating element fixed on the back surface of the support; and
a device for fixation of the light generating element on the back surface,
wherein the support includes at least three openings placed at predetermined positions on the support,
wherein the light generating element is fixed on the back surface of the support by the device for fixation to cover the openings such that the light generating element is detectable through the openings by the tracking system, and
wherein the light generating element can be placed or removed or replaced during an operation with the device for fixation.

2. The marker as defined in claim 1, wherein the openings are circular with a chamfer being located around the openings, a sharp part of the chamfer being close to the light generating element in order to reduce the tracking error when the marker is angulated.

3. The marker as defined in claim 1, wherein the light generating element covers at least one of the openings.

4. The marker as defined in claim 1, wherein the support comprises one light generating element per opening.

5. The marker as defined in claim 1, wherein device for fixation of the light generating element to the support of the marker includes by a spring mechanism.

6. The marker as defined in claim 5, wherein the spring mechanism comprises a leaf spring,

7. The marker as defined in claim 1, wherein the fixation of the light generating element to the support of the marker includes a magnet.

8. The marker as defined in claim 5, wherein the spring mechanism is located on the marker or support.

9. The marker as defined in claim 5, wherein the spring mechanism is located on the light generating element.

10. The marker as defined in claim 1, wherein the device for fixation is partly located on a light generating element and partly on the support.

11. The marker as defined in claim 1, wherein the light generating element includes a reflective foil.

12. The marker as defined in claim 11, wherein the reflective foil is applied on the back surface of the support and covers at least the openings.

13. The marker as defined in claim 11, wherein the reflective foil is maintained on the back surface by a lower marker part which is held by the device for fixation.

14. The marker as defined in claim 1, wherein the light generating element comprises at least a diffuser and a light source.

15. The marker as defined in claim 14 wherein the light source comprises a LED mounted on a PCB.

16. A tracking system comprising a marker, the marker including,
a support with a front surface and a back surface;
a light generating element fixed on the back surface of the support; and
a device for fixation of the light generating element on the back surface,
wherein the support includes at least three openings placed at predetermined positions on the support,
wherein the light generating element is fixed on the back surface of the support by the device for fixation to cover the openings such that the light generating element is detectable through the openings by the tracking system, and
wherein the light generating element can be placed or removed or replaced during an operation with the device for fixation.

17. A computer assisted surgery system comprising at least a marker, the marker including,
a support with a front surface and a back surface;
a light generating element fixed on the back surface of the support; and
a device for fixation of the light generating element on the back surface,
wherein the support includes at least three openings placed at predetermined positions on the support,
wherein the light generating element is fixed on the back surface of the support by the device for fixation to cover the openings such that the light generating element is detectable through the openings by the tracking system, and
wherein the light generating element can be placed or removed or replaced during an operation with the device for fixation.

18. The computer assisted surgery system as defined in claim 17, further comprising a robotic element.
